(19) United States
(12) Patent Application Publication
(10) Pub. No.: US 2020/0199289 A1
(43) Pub. Date: Jun. 25, 2020

(54) SURFACE MODIFICATION OF SUBSTRATES USING POLY (2-OXAZINE)S

(71) Applicant: UNIVERSITEIT GENT, Gent (BE)

(72) Inventors: Richard Hoogenboom, Terneuzen (NL); Bart Verbraeken, Lummen (BE); Edmondo Benetti, Zurich (CH)

(21) Appl. No.: 16/721,127

(22) Filed: Dec. 19, 2019

(30) Foreign Application Priority Data
Dec. 21, 2018 (EP) PCT/EP2018/086596

(51) Int. Cl. C08G 61/12; A61L 27/18; A61K 47/34

(52) U.S. Cl. CPC C08G 61/122; C08G 2261/3228; A61K 47/34; A61L 27/18

(57) ABSTRACT

The present disclosure relates to the surface modification of substrates using poly (2-oxazine)s. Specifically, the present disclosure provides the use one or more poly(2-alkyl-2-oxazine) or copolymers containing poly(2-alkyl-2-oxazine) in surface modification of a substrate. More in particular the present disclosure provides substrates having attached thereto, or associated therewith, one or more poly(2-alkyl-2-oxazine) or copolymers containing poly(2-alkyl-2-oxazine); wherein said alkyl is preferentially selected from the list comprising methyl and ethyl. The present disclosure also provides methods for preparing such substrates and uses thereof.

### CROSS-REFERENCE TO RELATED APPLICATION

This application claims the benefit of priority under 35 U.S.C. § 119(a) to International Application No. PCT/EP2018/086596, filed Dec. 21, 2018, which application is hereby incorporated herein by reference in its entirety.

### TECHNICAL FIELD

The present disclosure relates to the surface modification of substrates using poly(2-oxazine)s. Specifically, the present disclosure provides the use one or more poly(2-alkyl-2-oxazine) or copolymers containing poly(2-alkyl-2-oxazine) in surface modification of a substrate. More in particular the present disclosure provides substrates having attached thereto, or associated therewith, one or more poly(2-alkyl-2-oxazine) or copolymers containing poly(2-alkyl-2-oxazine); wherein said alkyl is preferentially selected from the list comprising methyl and ethyl. The present disclosure also provides methods for preparing such substrates and uses thereof.

### BACKGROUND

Biopassivity and lubrication of molecularly tailored surfaces have been recently gaining particular industrial interest, both of these characteristics being fundamental to the design of many biomedical and biomechanical devices, such as articular prosthesis, catheters, intraocular lenses or biosensors.

Since many inorganic and organic materials used for these applications present a negatively charged, oxide interface at physiological pH values, comb-like or graft-copolymers featuring a polycationic backbone and bioinert side chains represent a highly versatile, robust and broadly applicable solution to the fabrication of brush assemblies through simple dip-and-rinse processes, simultaneously preventing protein contamination and reducing friction.

In particular, poly(L-lysine)-graft-poly(ethylene glycol) (PLL-g-PEG) has been applied to form lubricious and antifouling PEG-brush coatings on metal oxide surfaces with low isoelectric points, as well as on polymeric supports, while analogous graft-copolymers featuring PEG-bioconjugates have been successfully employed to support cell adhesion and proliferation on a variety of surfaces.

Despite the broad applicability of PLL-g-PEG films, several drawbacks have been associated with the application of PEG derivatives, including their tendency to oxidative degradation to yield toxic compounds, and the expression of antibodies specific for PEGs in vivo. Appropriate alternatives that display improved chemical stability would be highly desirable, while polymers presenting more easily tailorable chemistries would give access to a larger variety of functional surfaces.

### SUMMARY

In a first aspect, this disclosure provides a substrate having attached thereto, or associated therewith, one or more poly(2-alkyl-2-oxazine) or copolymers containing poly(2-alkyl-2-oxazine); wherein said alkyl is selected from the list comprising methyl and ethyl.

In a particular embodiment, said poly(2-alkyl-2-oxazine) are selected from the list comprising poly(2-methyl-2-oxazine), poly(2-ethyl-2-oxazine) or copolymers thereof.

In another particular embodiment, said copolymers are copolymers of poly(2-alkyl-2-oxazine) and poly(2-alkyl-2-oxazoline).

In yet a further embodiment, the minimum amount of poly(2-alkyl-2-oxazine) in said copolymers is 50 mol %

In a further embodiment, said substrate is selected from the list comprising: polymeric supports, metal and metal oxide supports, glass and quartz supports and silicon supports; alternatively said substrate may also be selected from the list comprising: (bio)medical implants, drug delivery carriers, biosensors, and marine coatings.

The present disclosure also provides the use of a substrate as defined herein in human or veterinary medicine. More in particular, the present disclosure provides the use of a substrate as defined herein in anti-biofouling, blood half-life extension, or lubrication. For blood half-life extension the poly(2-alkyl-2-oxazine) itself may act as substrate for coupling or loading or drugs.

In a further aspect, the present disclosure provides the use of one or more poly(2-alkyl-2-oxazine) or copolymers containing poly(2-alkyl-2-oxazine) in surface modification of a substrate.

Additional features and advantages of the embodiments described herein will be set forth in the detailed description which follows, and in part will be readily apparent to those skilled in the art from that description or recognized by practicing the embodiments described herein, including the detailed description which follows, the claims, as well as the appended drawings.

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this specification. The drawings illustrate the various embodiments described herein, and together with the description serve to explain the principles and operations of the claimed subject matter.

### BRIEF DESCRIPTION OF THE DRAWINGS

With specific reference now to the figures, it is stressed that the particulars shown are by way of example and for purposes of illustrative discussion of the different embodiments of the present disclosure only. They are presented in the cause of providing what is believed to be the most useful and readily description of the principles and conceptual aspects of the disclosure. In this regard no attempt is made to show structural details in more detail than is necessary for a fundamental understanding. The description taken with the drawings making apparent to those skilled in the art how the several forms may be embodied in practice.

FIG. 1 is a schematic diagram of surface functionalization by brush-forming graft copolymers presenting different side-chain composition.

FIG. 2 is a graph of force-vs-separation (FS) profiles recorded by AFM on the different brush layers.

FIG. 3A is a graph of protein adsorption from 10% HS on PLL-g-X films measured by VASE after 1 hour of exposure. In the graph, (*) p<0.05; and (**) p<0.01.

FIG. 3B is a graph of protein adsorption from 10% FBS on PLL-g-X films measured by VASE after 1 hour of exposure. In the graph, (*) p<0.05; and (**) p<0.01.

FIG. 3C is a plot of amount of adsorbed proteins as a function of polymer brush hydration (H₂O/monomer) as determined from the experiments of FIGS. 3A and 3B.

FIG. 4A is a graph of bovine chondrocyte adhesion on the different PLL-g-X films after 24 hours of culture, with complemented 10% FBS.

FIG. 4B is a graph of bovine chondrocyte adhesion on the different PLL-g-X films after 24 hours of culture, without complemented 10% FBS.

FIG. 4C is a plot correlating percent of adhered cells to brush hydration (H₂O/monomer) in the determination of bovine chondrocyte adhesion on the different PLL-g-X films after 24 hours of culture graphed in FIGS. 4A and 4B.

FIG. 5 is a bar graph of cytotoxicity of the different copolymer side-chains dissolved at increasing concentrations in cell-culture media. Bovine chondrocytes were exposed for 24 hours to the polymer solutions and their viability was measured through a LIVE/DEAD protocol. Mean values and standard deviations were calculated from three replicates and normalized to the control (bovine chondrocytes exposed to cell culture medium).

FIG. 6 is a graph of FfL profiles recorded by LFM on different brush films.

### DETAILED DESCRIPTION

The era of the poly(ethylene glycol) (PEG) brushes as a universal panacea for resisting non-specific protein adsorption and providing lubrication to surfaces is approaching an end. Intensive efforts to find alternative, more stable polymer-brush systems for the tailoring of biomaterials are underway. In the functionalization of medical devices and implants, in addition to preventing non-specific protein adsorption and cell adhesion, polymer-brush formulations are often required to generate highly lubricious films.

As alternatives to the PEG brushes, poly(2-alkyl-2-oxazoline) (PAOXA) brushes meet these requirements. By tailoring the composition of their side groups they can form films that meet or surpass the bioinert and lubricious properties of PEG analogues. Poly(2-methyl-2-oxazine) (PMOZI), which is isomeric to, but structurally different from poly(2-ethyl-2-oxazoline) (PEOXA), provides an additional enhancement of brush hydration and main-chain flexibility, leading to complete bioinertness and a further reduction of friction.

Poly(2-alkyl-2-oxazoline)s (PAOXAs), in particular the hydrophilic poly(2-methyl-2-oxazoline) (PMOXA) and poly(2-ethyl-2-oxazoline) (PEOXA), are believed to be promising alternatives to PEGs in a variety of biotechnological applications, showing comparable physicochemical properties, high biocompatibility, and stealth properties. Moreover, PAOXA films on organic and inorganic supports show similar biopassivity to their PEG counterparts, and a significantly higher resistance toward oxidation. Furthermore, poly(2-alkyl-2-ozazine) (e.g. PAOZI) grafts may also be a highly valuable alternative for surface modification. In particular, poly(2-methyl-2-oxazine) (PMOZI) is isomeric to PEOXA, but presents a methyl group as a side chain (as in PMOXA) and contains one additional methylene function along the repeating unit.

As with PAOXAs, poly(2-alkyl-2-oxazine)s (PAOZIs) are synthesized by cationic ring-opening polymerization (CROP), starting from 5-membered or 6-membered cyclic imino ether monomers, respectively, enabling a controlled polymerization process under mild conditions and the accessible preparation of multifunctional polymers. Since PMOZI features very similar chemical traits to PMOXA and PEOXA but has a different structural arrangement within the monomer unit, it represents a particularly interesting polymer to evaluate the effects of the macromolecular architecture on the physicochemical properties of the subsequently generated brushes.

For antifouling it is generally believed that the more hydrophilic the polymer, the more effective the antifouling properties will be. As such, a person skilled in the art would expect the following order in antifouling efficiency: PMOXA>PMOZI>PEOXA. Even so, it is unexpectedly found that PMOZI coatings are better antifouling than PMOXA.

As already indicated herein above, the present disclosure provides a substrate having attached thereto, or associated therewith, one or more poly(2-alkyl-2-oxazine) or copolymers containing poly(2-alkyl-2-oxazine); wherein said alkyl is selected from the list comprising methyl and ethyl.

The term “alkyl” by itself or as part of another substituent refers to a fully saturated hydrocarbon of Formula CₓH₂ₓ₊₁ wherein x is a number greater than or equal to 1. Generally, alkyl groups of this disclosure comprise 1 or 2 carbon atoms. When a subscript is used herein following a carbon atom, the subscript refers to the number of carbon atoms that the named group may contain. Thus, for example, C₁₋₂alkyl means an alkyl of one to two carbon atoms. Examples of alkyl groups of the present disclosure are methyl and ethyl.

In the context of the present disclosure, the term ‘attached to’ is meant to represent a covalent link between said substrate and said poly(2-alkyl-2-oxazine) or copolymers comprising the same. Alternatively, non-covalent interaction between said substrate and poly(2-alkyl-2-oxazine) or copolymers comprising the same may also occur, herein being termed ‘associated with’.

In the context of the present disclosure, the term poly(2-alkyl-2-oxazine) is generally represented according to formula (I) as follows:

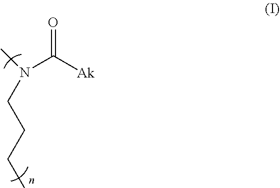

In formula (I), Ak represents any alkyl moiety; and n may be any integer, typically from 10 to 200; more in particular about 50 to 100. In formula (I), specific examples of Ak in embodiments include methyl and ethyl moieties, as in the following:

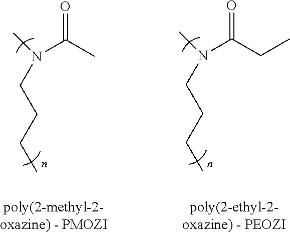

Hence, in a particular embodiment, said poly(2-alkyl-2-oxazine) are selected from the list comprising poly(2-methyl-2-oxazine), poly(2-ethyl-2-oxazine) or copolymers thereof.

In another particular embodiment, said copolymers are copolymers of poly(2-alkyl-2-oxazine) and poly(2-alkyl-2-oxazoline). In the context of the present disclosure said poly(2-alkyl-2-oxazoline) are represented as formula (II):

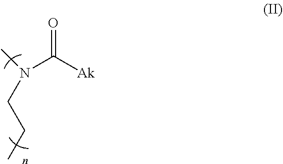

In formula (II), Ak represents any alkyl moiety; and n may be any integer, typically from 10 to 200; more in particular about 50 to 100. Specific non-limiting examples of Ak in formula (II) include methyl and ethyl.

While the copolymers of embodiments herein may contain any amount of poly(2-alkyl-2-oxazine), they preferably contain at least 50 mol % of poly(2-alkyl-2-oxazine). In the context of the present disclosure any reference to mol % is meant to be with respect to the total (co-)polymer. Hence, where a particular component (such as poly(2-alkyl-2-oxazine)) constitutes 50 mol % of the (co-)polymer, the remaining 50 mol % of the (co-)polymer may be made up of different components such as poly(2-alkyl-2-oxazoline).

The compounds of the present disclosure can be made using conventional techniques. An exemplary reaction scheme is shown in the Examples Section. Typically, an oxazine, particularly a 2-oxazine that includes an alkyl group at the 2-position, is subjected to a ring opening reaction in a suitable solvent (e.g. acetonitrile) in the presence of an initiator (e.g., methyl trifluoromethansulfonate (i.e.. methyl triflate), perfluorobutyl ethylene triflate, perfluorobutyl sulfonamide triflate, methyl toluene sulfonate (i.e., methyl tosylate), and methyl iodide) with heating (e.g., at a temperature of 80° C. or 140° C.), and subsequently modified to include a reactive group required for conjugation to the surface (eg dopamine) or coupling to a polymer (eg polylysine) to induce interaction with the surface. The reactive group can be incorporated in the PAOZi through the use of functional initiators, functional terminating agents or copolymerization with functional monomers as known in the state-of-the-art.

The polyoxazine (co-)polymers of the present disclosure are useful in making biofilm-resistant coatings (i.e., coatings that resist biofilm formation and/or enhance the release of formed biofilms, as evidenced by resistance to the growth of at least one microorganism). Thus, methods of coating a substrate to improve biofilm resistance of the substrate (relative to the uncoated substrate) are provided by the present disclosure. In one embodiment, a coating composition is provided that includes a polyoxazine (co-)polymer of the present disclosure and a solvent, whereby the coating compositions are applied to substrates to impart a biofilm-resistant coating thereto.

In another embodiment, there is a method for coating a substrate, with a coating composition of the present disclosure to provide a biofilm-resistant coating thereto. A wide variety of coating methods can be used to apply a composition of the present disclosure, such as brushing, spraying, dipping, rolling, spreading, and the like.

Useful solvents for the coating compositions include any that do not deleteriously affect polymerization of the monomers (if the coating solvent is the same as used in the polymerization process) and in which the components are soluble to at least 1% by weight. Examples of solvents are water, methanol, ethanol, isopropanol, acetone, methyl ethyl ketone, methyl iso-butyl ketone, methyl acetate, ethyl acetate, heptane, toluene, xylene, and ethylene glycol alkyl ether. Those solvents can be used alone or as mixtures thereof.

The coating composition is typically a homogeneous mixture that has a viscosity appropriate to the application conditions and method. For example, a material to be brushed or roller coated would likely be preferred to have a higher viscosity than a dip coating solution. The coating composition is typically a relatively dilute solution, often containing at least 0.1 wt %, or at least 1 wt %, of the (co-)polymer. A typical coating composition often contains no more than 50 wt %, or no more than 25 wt %, of the (co-)polymer.

The substrate on which the coating can be disposed for the formation of a biofilm-resistant coating can be any of a wide variety of materials. Useful substrates include ceramics, siliceous substrates including glass, metal, natural and man-made stone, woven and nonwoven articles, polymeric materials, including thermoplastics and thermosets, including, for example, poly(meth)acrylates, polycarbonates, polystyrenes, styrene copolymers such as styrene acrylonitrile copolymers, polyesters, polyethylene terephthalate, silicones such as that used in medical tubing, paints such as those based on acrylic resins, powder coatings such as polyurethane or hybrid powder coatings, and wood. The substrate can be in the form of a film, woven, or nonwoven, for example. In a very specific embodiment, the substrate is selected from the list comprising: polymeric supports, metal and metal oxide supports, glass and quartz supports and silicon supports; alternatively said substrate may also be selected from the list comprising: (bio)medical implants, drug delivery carriers, biosensors, and marine coatings.

Various articles can be effectively treated with the coating composition of the present disclosure to provide a biofilm-resistant coating thereon. The present disclosure also provides a coated article, such as a film. Thus, the present disclosure provides an article comprising a substrate (e.g., a film), wherein the substrate includes at least one surface having a layer that includes a (co)polymer of the present disclosure disposed thereon.

Preferably, the substrate to which coating is to be applied should be clean prior to application to obtain optimum characteristic and durability. Metallic as well as glass surfaces are often covered with organic contaminants. Before the coatings of the present disclosure can be applied to such surfaces, they should he cleaned by at least solvent wiping. In the case of gross contamination, the metallic or glass surface may have to be etched, anodized, or treated in ways known to those skilled in the art. For example, if the surface of steel is coated with rust, that rust may have to be etched away by an acid treatment. Once the surface of the metal is exposed, the coating can be applied.

Biofilms typically develop where the substrate is in contact with water or exposed to humid conditions. The coatings of the present disclosure retard the formation of such biofilms, particularly when exposed to circulating water. It is believed that the microorganisms are unable or minimally able to attach to the coated surfaces. Further, it is believed that extant biofilms are more easily removed from the coated surface. Thus, the compositions of the present disclosure are particularly suited for substrate in wet or humid environments such as in medical catheter coatings, antifouling marine coatings, coatings for water handling equipment, heat exchangers and other HVAC equipment, coatings for filter media, and dental equipment, devices and materials that may be used in the oral cavity.

The present disclosure thus also provides the use of a substrate as defined herein in human or veterinary medicine. More in particular, the present disclosure provides the use of a substrate as defined herein in anti-biofouling, blood half-life extension, or lubrication.

In a further aspect, the present disclosure provides the use of one or more poly(2-alkyl-2-oxazine) or copolymers containing poly(2-alkyl-2-oxazine) in surface modification of a substrate.

### EXAMPLES

Embodiments of the present disclosure will be better understood by reference to the following examples, which are offered by way of illustration and which one skilled in the art will recognize are not meant to be limiting.

### Methods

Determination of Copolymer Side-Chain Density

The number of side chains per lysine unit (X/Lys) was determined by ¹H-NMR spectroscopy. In particular, after functionalization of the PLL backbone with PMOXA, PEOXA, PEG and PMEOZI, the signals corresponding to both the PLL and the side chains were found in the NMR spectra. It is noteworthy that the peak corresponding to the lysine methylene unit next to the terminal amino group was shifted from 3.0 to 3.15 ppm due to amide formation during the side-chain grafting. By integrating both peaks (the unmodified —CH₂-NH₂ and —CH₂—N(C═O)—), the grafting density was calculated as I_{d}/(I_{b}+I_{d}).

Proton Nuclear Magnetic Resonance Spectroscopy (¹H-NMR)

¹H-NMR spectra were recorded on a Bruker Avance III 700 MHz spectrometer at room temperature using D₂O as solvent.

Size Exclusion Chromatography (SEC)

Number- and weight-average absolute molecular weights, Mₙ and M_{w}, of PAOXA samples were determined using an Agilent 1100 SEC unit equipped with two PFG linear M columns (PSS) connected in series with an Agilent 1100 VWD/UV detector operated at 290 nm, a DAWN HELEOS 8 multi-angle laser-light-scattering (MALS) detector (Wyatt Technology Europe) followed by an Optilab T-rEX RI detector from Wyatt. Samples were eluted in hexafluoroisopropanol (HFIP) with 0.02 M K-TFAc at 1 mL/min at room temperature.

Absolute molecular weights were evaluated with Wyatt ASTRA software and dn/dc values based on our analytical setup (dn/dc (PEOXA)=0.2284 mL/g; dn/dc (PMOXA)=0.2498 mL/g; dn/dc (PMeOZI)=0.0808 mL/g).

Preparation of PLL-g-X Films

Silicon wafers (Si-Mat, Germany) were cleaned for 15 minutes in a Piranha solution (3:1 mixture of concentrated H₂SO₄ and H₂O₂), extensively washed with ultra-pure water and finally dried under a stream of nitrogen gas. Freshly cleaned wafers were immersed overnight in 0.1 mg/mL HEPES I (10 mM 4-(2-hydroxyethyl)-1-piperazineethanesulfonic acid, adjusted to pH 7.4) solutions of PLL-x, washed extensively with ultra-pure water and finally dried under a stream of N₂.

Variable Angle Spectroscopic Ellipsometry (VASE)

PLL-x film dry thickness was measured using a M-2000F variable-angle spectroscopic ellipsometer from J. A. Woollam Co (Lincoln, Nebr., USA). All data were recorded at a wavelength range between 370 and 1000 nm using focusing lenses at 70° from the surface normal. The raw ellipsometric data were analyzed with WVASE32 software using a three-layer model (Si/Si₂O/Cauchy; Aₙ=1.45 and Bₙ=0.01, Cₙ=0). All measurements were performed under ambient conditions. Three substrates were prepared for each PLL-x film and five points were measured on each sample to calculate the mean values and standard deviations.

Quartz Crystal Microbalance with Dissipation (QCM-D) PLL-x film hydrated thickness was measured by QCM-D using an E4 instrument (Q-Sense AB, Goteborg, Sweden) equipped with dedicated Q-Sense AB software. SiP₂-coated crystals (LOT-Oriel AG) with a fundamental resonance frequency of 5 MHz were used as substrates.

Before the experiment, the substrates were cleaned by sonication in toluene and 2-propanol and UV-ozone treatment (UV Clean Model 135500 from Boekel Industries, Inc.). After cleaning, the crystals were dried under a stream of N₂ and used immediately. The crystals were exposed to ultrapure water at 25° C. until a stable baseline was established. Later, the water was replaced with HEPES I buffer until a new baseline was reached. A 0.1 mg mL⁻¹ PLL-g-X solution in HEPES I was then injected until complete adsorption was obtained. To determine the stability of the film and eliminate the physisorbed polymers, washing steps were performed with HEPES I and ultrapure water. The values of hydrated thickness for the different PLL-x films were obtained by applying a Voigt extended viscoelastic model to fit the frequency and dissipation shift, using three overtones (5th, 7th and 9th). Two crystals for each PLL-x film were used to calculate the mean values and standard deviations.

Atomic Force Microscopy (AFM)

Friction and normal force measurements were carried out by atomic force microscopy (AFM) and lateral force microscopy (LFM), respectively, using a MFP3D AFM (Asylum Research, Oxford Instruments, Santa Barbara, USA) under HEPES I buffer (pH=7.4). The normal (K_{N}) and the torsional (K_{T}) spring constants (KN=0.107 N m⁻¹ and KT=2.49E-9 N m) of tipless cantilevers (CSC38/tipless/Cr-Au, Mikromash, Bulgaria) were measured by the thermal noise and Sader's method, respectively, prior to attachment of 20-μm-diameter silica spheres to the cantilevers. The colloidal probes were prepared by gluing the silica particle (EKA chemicals AB, Kromasil R, Sweden) to the end of a tipless cantilever using a home-built micromanipulator.

Friction-force values were obtained by averaging approximately 10 measured “friction loops” across each film. The friction loops were acquired by laterally scanning the cantilever on a single line for each applied load over 2 different positions on the investigated sample. A sliding distance of 2 μm and a scanning speed of 2 μm s⁻¹ were used for the measurements. Normal-force measurements were carried out with the same cantilevers used for the friction tests. Approximately 40 force-vs-separation (FS) curves were acquired over 2 different positions on each sample. A scanning distance of 0.5 μm with a scanning speed of 0.5 μm s⁻¹ were used to obtain the FS curves.

Water-Contact-Angle Measurements (CA)

Surface wettability was determined by dynamic water contact angle (DSA 100, Krüss, Hamburg, Germany) in an automated procedure. For measuring the advancing contact angles, the volume of dispensed water drop was increased from 4 μL to 10 μL in two steps. Videos of 175 frames were recorded for each step. For measuring the receding contact angles, the drop volume was decreased from 10 μL to 0 μL in one step with video recording of 500 frames. The speed of all measurements was 15 μL min⁻¹. Three different locations were measured on each sample. For evaluation, video sequences of moving drops were fitted with a tangent method 2fit routine, using a fourth-order polynomial function (Drop-Shape Analysis program, DSA3 software, Krüss).

Protein Adsorption Studies by VASE

The resistance towards protein adsorption by the different PLL-g-X films was assessed by VASE. PLL-g-X adlayers deposited on silicon wafers were immersed for 5 minutes in ultrapure water to hydrate the polymer assemblies. After this time, the water was removed and PLL-g-X films were exposed to 10% human serum (HS) or 10% fetal bovine serum (FBS) at room temperature for 1 hour. During incubation, the samples were kept under ambient conditions without stirring. Following exposure to serum solutions, the samples were rinsed with ultrapure water, dried under a stream of N₂ and the remaining adsorbed serum was measured by VASE. Bare silicon wafers were used as positive controls. Three substrates for each PLL-x film were prepared and five points for each sample were measured to calculate the mean values and standard deviations.

Cell-Adhesion Tests

PLL-x films on silicon wafers were transferred inside 24-well plates and seeded with bovine chondrocytes at passage 2 (10000 cells/cm²). After 24 h of incubation at 37° C. in cell culture media, the samples were washed with PBS and fixed with a 4% paraformaldehyde +0.1% Triton-X solution for 20 minutes at 4° C. After washing with PBS (2 times), the samples were incubated at room temperature with rhodamine-labelled phalloidin/DAPI (130 ng/mL and 0.3 μM, respectively) for 30 minutes. The samples were washed with PBS and mounted on a coverslip with aqueous mounting media. Three images were acquired for PLL-x films with a Zeiss ApoTome.2 fluorescence microscope and image analysis was performed using Fiji software. All the experiments were done in triplicate and bare substrates were used as positive controls.

All the experiments were done in triplicate, and bare substrates were used as positive controls. To assess the role of FBS on the cell adhesion, the same experiment was performed using cell culture media without FBS.

In Vitro Cytotoxicity Studies

To assess the cytotoxicity of the PLL-x side chains (COOH-terminated PMOXA, PEOXA, PEG, PMeOZI), primary bovine chondrocytes at passage 2 (10'000 cells/cm²) were seeded in 12-well plates and expanded for 24 h. After this time, the cell-culture medium (Dulbecco's modified Eagle's medium (DMEM-31966), 10% Fetal Bovine Serum (FBS), 50 μg/mL L-ascorbic acid, and 1% penicillin-streptomycin) was replaced with solutions of PMOXA, PEOXA, PEG and PMEOZI at 5 different concentrations (250, 100, 50, 10, 1 mg/mL in cell culture medium). The plates were then incubated for 24 h at 37° C. Chondrocyte viability was tested using the LIVE/DEAD cell viability assay. Briefly, the plates were incubated with 0.66 μg/ml propidium iodide, 0.5 μg/ml calcein AM and 10 μg/ml Hoechst 33342 in plain DMEM at 37° C. for 30 minutes. Afterwards, they were washed twice with PBS and once with medium. All the experiments were done in triplicates and cells exposed to cell culture medium only were used as control. Two images per scaffold were acquired with a Zeiss ApoTome.2 fluorescence microscope and image analysis was performed using Fiji software.

### Statistical Analysis

Statistical evaluation was performed with IBM SPSS Statistics (version 24), using a one-way ANOVA, with Tukey's post-hoc test to assess the differences between the properties of PLLx films. p<0.05 was noted with an asterisk (*), p<0.01 with two asterisks (**), otherwise with the exact number.

### Synthesis of Polymers

The overview of the synthesis of PLL-PMOZI is depicted in the scheme below. Further experimental details are provided in the following part.

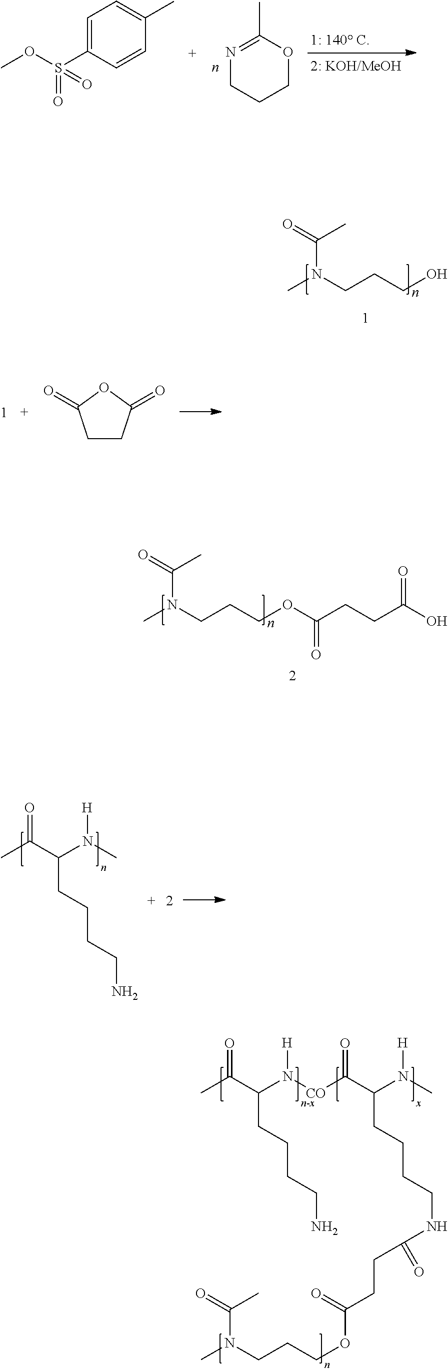

Top: Synthesis for PMOZI-OH; middle: Synthesis of PMOZI-COOH; bottom: Synthesis of PLL-PMOZI.

Synthesis of hydroxyl-terminated poly(2-methyl-2-oxazine) (PMOZI-OH)

3-Amino-1-propanol (150 mL, 1.97 mol, 1.1 eq.) was added to a suspension of Zn(OAc)2.2H2O (7.86 g, 35.8 mmol, 0.02 eq.) in ACN (93.68 mL, 1.79 mol, 1.0 eq.). After refluxing for 72 h the crude product was purified through fractional distillation (132° C., atmospheric pressure) and 2-methyl-2-oxazine (MOZI) was obtained as colourless liquid (88.47 g, 45%).

Acetonitrile (9.2 mL), MOZI (5.6mL, 60 mmol, 100 equiv.) and MeOTs (90.5 μL, 0.06 mmol, 1 equiv.) were mixed in a Biotage microwave vial (20 mL). The solution was polymerized in a microwave oven for 50 minutes at 140° C. After this time, the polymerization was terminated by addition of a 1.0 M solution of KOH in methanol (0.30 mL) at 0° C., and left stirring overnight at room temperature. The polymer product was purified by precipitation in cold diethyl ether and subsequently dried overnight under vacuum, to finally yield a sticky, yellowish solid (3.7 g).

PMEOXA-OH and PEOXA-OH were made in a similar manner using MEOXA and EOXA as monomers instead of MEOZI.

Synthesis of Acid-Terminated poly(2-methyl-2-oxazine) (PMOZI-COOH)

To convert PMOZI-OH into PMOZI-COOH, PMOZI-OH (1 eq) and succinic anhydride (10 eq) were dissolved in dry ACN and refluxed at 90° C. overnight. The final product was obtained after solvent removal, dissolution in MilliQ water and purification via dialysis (SpectraPor, MWCO 1 kDa, 24 h against NaCl solution, 24 h against 10% acetic acid solution and 24 h against milliQ water). After freezedrying, PMOZI-COOH was obtained as a white solid.

PMOXA-COOH and PEOXA-COOH were made in a similar manner using PMOXA-OH and PEOXA-OH instead of PMOZI-OH.

Grafting Different Side Chains onto PLL

PLL-g-X graft-copolymers (with X=PMOXA-COOH, PEOXA-COOH, PMOZI-COOH or PEG-COOH (purchased)) were synthesized according to the already reported procedure (Konradi et al., 2008). Poly-L-lysine hydrobromide (100 mg, 0.48 mmol of NH₃⁺, MW 15000-30000 g/mol, Sigma-Aldrich), the selected side chain X (0.16 mmol, corresponding to 0.33 X/Lysine unit), N-hydroxysulfosuccinimide sodium salt (sulfo-NHS, 0.16 mmol) and N-(3-dimethylaminopropyl)-N′-ethylcarbodiimide hydrochloride (EDC, 1.6 mmol) were dissolved separately in 2 mL of 10 mM 4-(2-hydroxyethyl)-1-piperazineethanesulfonic acid (HEPES) buffer solution (pH 7.4), and mixed in the order given above. The mixture was left under stirring overnight and purified via dialysis (SpetraPor, MWCO 12000-14000 kDa) for 2 days. PLL-g-X were obtained after freeze-drying as white solids.

### Evaluation of Antifouling and Lubrication Properties

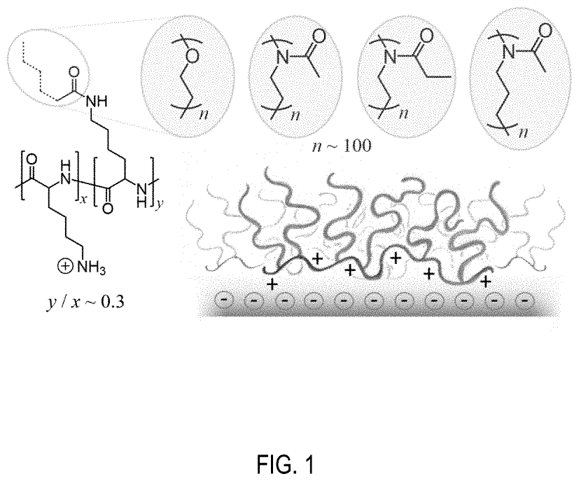

In this study, we systematically compare the interfacial physicochemical properties of brushes generated from graft-copolymers PLL-g-PMOXA, PLL-g-PEOXA and PLL-g-PMOZI on SiO2 surfaces, and correlate them to those displayed by PLL-g-PEG (FIG. 1). Particular attention is paid to the structural characteristics of the chemically different brush layers, their hydration, as well as their biopassive and nanotribological properties, which were analyzed by a combination of surface-sensitive techniques, including variable angle spectroscopic ellipsometry (VASE), quartz crystal microbalance with dissipation (QCM-D) and atomic force microscopy (AFM)-based methods. All these characteristics, are extremely relevant when such brush films are applied on medical devices and implants that require both surface biopassivity and lubrication.

All the graft-copolymers (PLL-g-X) employed for surface functionalization featured side chains with degree of polymerization (DP) ˜100, and side-chain densities on the PLL “backbone” of ˜0.3 chains per lysine unit (X/Lys in Table 1). This particular value of X/Lys was chosen to guarantee a relatively high concentration of positively charged ammonium groups on the PLL, which drive the surface assembly, as well as a high enough side-chain loading to enable the formation of a uniform and dense brush film.

TABLE 1
Characterization of PLL-g-X films by VASE, QCM-D and CA

| | | T_{dry} | σ | L | | T_{wet} | H₂O/ | Contact angle |
|---|---|---|---|---|---|---|---|---|
| PLL-g-X | X/Lys | (nm) | (X nm⁻²) | (nm) | L/2R_{g} | (nm) | monomer | Adv./Rec. (°) |
| PLL-g-PMOXA | 0.31 | 1.3 ± 0.1 | 0.09 | 3.6 | 0.37 | 8.9 ± 1.7 | 26 ± 3 | 10/6 |
| PLL-g-PEOXA | 0.33 | 1.3 ± 0.2 | 0.07 | 4.1 | 0.39 | 5.1 ± 1.2 | 14 ± 4 | 35/26 |
| PLL-g-PMOZI | 0.32 | 1.5 ± 0.1 | 0.10 | 3.3 | 0.35 | 9.2 ± 1.9 | 25 ± 3 | 13/8 |
| PLL-g-PEG | 0.34 | 1.3 ± 0.1 | 0.16 | 2.6 | 0.47 | 9.9 ± 1.3 | 19 ± 2 | 30/18 |

VASE showed a similar dry thickness (T_{dry}, in Table 1) for all the graft-copolymer films, with values included between 1.3 and 1.5 nm (see Supporting Information for details). The values of T_{dry} were used to estimate the surface grafting density (σ) for each brush type, the distance between grafting points (L) and the degree of chain overlap at the surface (L/2R_{g}). PMOXA, PEOXA and PMOZI brushes all presented similar values of σ, ranging from 0.07 to 0.10 chains nm⁻², whereas PEG grafts featured a slightly higher surface density of 0.16 chains nm⁻², presumably due to the lower molar mass and molecular dimensions of PEG (M_{w}˜5 kDa) with respect to PAOXAs and PMOZI with comparable DP (M_{w} ranging from 9 to 11 kDa), which enabled the assembly of a higher graft-copolymer concentration at the surface. Despite the different values of a, the assemblies showed a similar degree of chain overlap, in all cases less than 1, suggesting analogous brush configuration and morphology irrespective of the grafts' compositions.

The combination of QCM-D and VASE data allowed us to further compare the hydration properties of the different brushes. As highlighted in Table 1, PMOXA and PMOZI brushes showed the highest concentration of water molecules per monomer unit (indicated as H₂O/monomer), 26±3 and 25±3, respectively, whereas brush hydration progressively decreased for PEG and PEOXA brushes (H₂O/monomer=19±2 and 14±4, respectively). The lower hydrophilicity of PEG and PEOXA brushes compared to PMOXA and PMOZI analogues was confirmed by water-contact-angle measurements (CA), which showed significantly higher values of advancing and receding CA for the two former films. Interestingly, the markedly hydrophilic character of PMOZI brushes is in agreement with the solution properties of this polymer, which, while isomeric with PEOXA does not display a lower critical solution temperature (LCST), in a similar way to PMOXA. Hence, the composition of the side groups appears to be the predominant factor determining polymer hydration, rather than the chemical nature of the main chain.

PEOXA grafts showed the most hydrophobic character, as indicated by their limited swelling in water and relatively high values of advancing and receding CA.

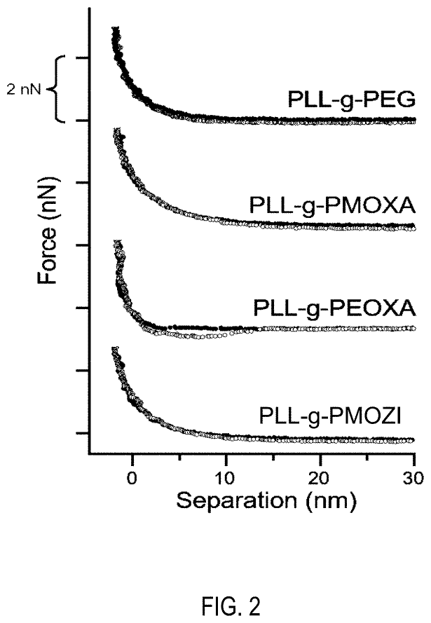

This behavior was further confirmed by analysing the adhesive properties of the layers by AFM. As reported in FIG. 2, PMOXA, PMOZI and PEG brushes displayed marked repulsive interactions with the AFM colloidal silica probe, with both approaching and retracting profiles nearly overlapping each other. In contrast, the force-vs-separation (FS) profile recorded on PEOXA-based films showed a “jump-in” along the approaching, and adhesive interactions along the retracting curve, suggesting the presence of attractive, van der Waals forces between the silica colloid and PEOXA chains.

The physicochemical characteristics of the different brushes were mirrored by a varied resistance towards protein adsorption from solution. The biopassivity of PLL-g-X films was tested, alternatively subjecting them to 10% human (HS) and fetal bovine serum (FBS) for 1 hour, and subsequently measuring the amount of adsorbed proteins by VASE.

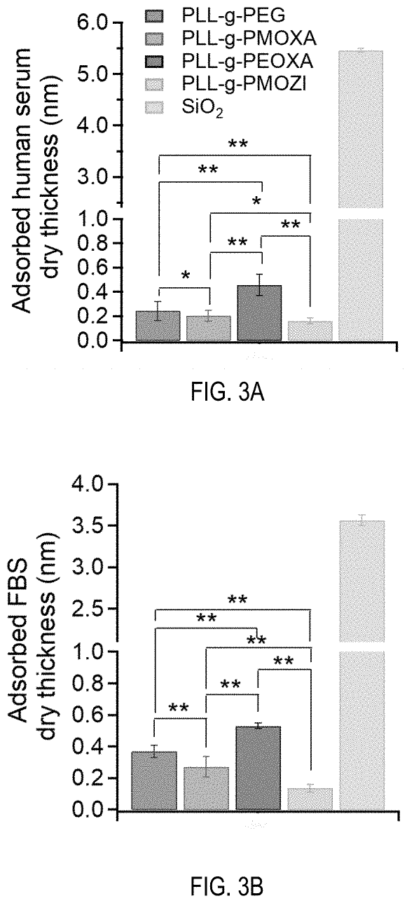

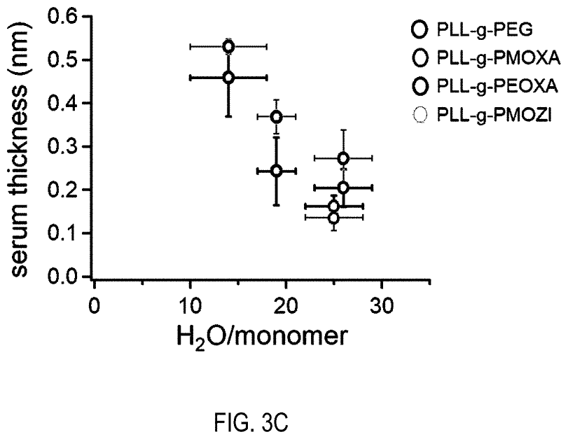

All the brushes significantly reduced the amount of adsorbed protein both from HS (FIG. 3A) and FBS (FIG. 3B), compared to the bare SiO₂ surface. However, PEOXA brushes were the least antifouling layers, with a 91% and 85% reduction of physisorbed serum, from HS and FBS, respectively. PEG and PMOXA brushes performed similarly, reducing protein contamination by more than 90%. Surprisingly, PMOZI brushes nearly quantitatively hindered protein adsorption from both HS and FBS, reaching 97% and 96% of adsorbed-serum reduction, respectively.

The resistance towards protein fouling correlated directly with brush hydrophilicity. As shown in FIG. 3C, the amount of adsorbed proteins decreased with the increasing concentration of water molecules per monomer unit, the most hydrophilic brushes (PMOXA and PMOZI) producing the best antifouling layers. However, the nearly quantitative resistance towards protein contamination displayed by PMOZI brushes could not be solely explained by polymer hydration, which was similar to that recorded for PMOXA grafts. Without intent to be bound by theory, it is believed that the higher flexibility of PMOZI chains with respect to its PAOXA counterparts, which is determined by the additional methylene group in the repeating unit along the PMOZI main chain, generates brushes that provide a more efficient entropic barrier towards approaching biomolecules. In support of this hypothesis, bulk PMOZI shows a glass transition (T_{g}) below ambient temperature, significantly lower than the T_{g} of the isomeric PEOXA (T_{g}˜60° C.).

Since brush-forming graft copolymers could be easily applied for the functionalization of implants, their integration within a surrounding tissue environment was further evaluated by testing the adhesion of bovine chondrocytes. In particular, preventing unspecific cell adhesion on these brush coatings would later allow us to introduce well-determined functionalities/peptide sequences that direct cell settlement and proliferation.

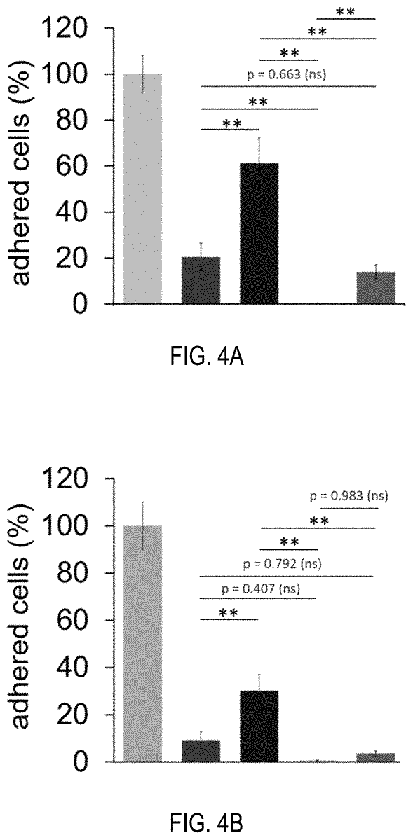

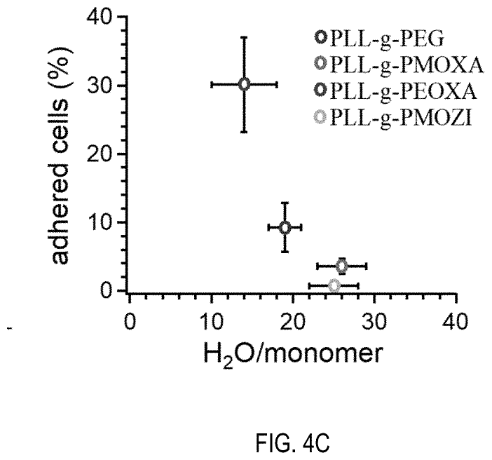

Relevantly, after 24 hours of incubation, PLL-g-PMOZI films had no cells attached, while PLL-g-PMOXA, PLL-g-PEG and PLL-g-PEOXA displayed the adhesion of 15, 20 and 60% of cells, respectively, when compared to bare SiO₂, which was chosen as a positive control (FIG. 4A). In a similar way, PLL-g-PMOZI quantitatively prevented the unspecific settlement of cells without complementing the culture medium with 10% FBS (FIG. 4B), while PLL-g-PMOXA, PLL-g-PEG and PLL-g-PEOXA analogues showed 2, 5 and 25% of adhered cells, respectively.

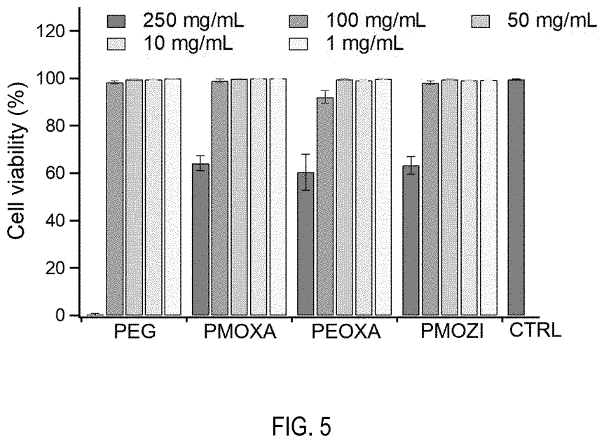

As with the biopassive character towards serum proteins, the combination of high hydration and chain flexibility by PMOZI brushes produced films that fully hindered cell adhesion (FIG. 4C). It is also relevant that these unique antifouling properties were not due to any cytotoxic character of the PMOZI side chains, as confirmed by biocompatibility tests (FIG. 5).

Besides their resistance against unspecific protein and cell adhesion, brush lubrication can be a fundamental requirement when these are applied on the exposed surface of medical devices.

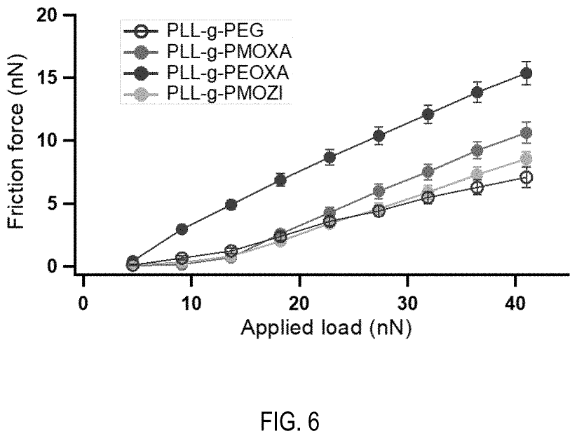

The nanotribological properties of PLL-g-X films were assessed by lateral force microscopy (LFM), recording friction force-vs-applied load profiles (FfL) on the different brush films. As displayed in FIG. 6, PEOXA brushes showed the highest friction among the different films studied, probably due to their limited hydration and amphiphilic character. In contrast, an improvement in the lubrication properties was found for PMOXA, PMOZI and PEG brushes, the latter two brush types displaying the lowest friction.

These results corroborated the direct correlation between biopassive and lubrication properties, which was previously found for different brush chemistries and structures, both these two characteristics being determined by brush surface density and polymer hydration. However, the lowest friction values recorded for PEG and PMOZI brushes suggested that when comparing the nanotribological properties of hydrophilic polymer grafts, those featuring higher chain flexibility are slightly more lubricious than more rigid brushes. In agreement with this assumption, the slope of FfL profiles progressively decreased with the T_{g} of the polymer, which correspond to 78° C. for PMOXA, 16° C. for PMOZI, and −35° C. for PEG.

In summary, the comparative analysis of PAOXA, PMOZI and PEG brushes, formed on SiO2 surfaces via graft-copolymer assembly, highlights how polymer hydration and flexibility determine the performance of the brush layers as lubricious biointerfaces. PAOXA brushes can match the biopassive and frictional properties of PEG analogues, and outperform the attractive properties of these latter films in the case of the most hydrophilic PMOXA. The presence of an additional methylene group within the polymer-repeating unit, as in the case of PMOZI brushes, maintains their hydration capabilities unaltered in comparison to PMOXA analogues, and substantially improves them with respect to the isomeric PEOXA grafts.

The combination of high hydration and enhanced chain flexibility, guaranteed by longer propyl segments spacing the amide moieties, substantially reduce friction and generate an exceptional enthalpic and entropic barrier against protein and cell adhesion. Moreover, while featuring an analogous composition to PAOXAs, PMOZI brushes are expected to feature a similarly improved chemical resistance towards oxidative degradation compared to PEGs, especially within physiological media. Hence, PMOZI emerges as a new polymer for the generation of brushes with unprecedented properties, significantly surpassing the state-of-the-art, and opening up a plethora of possible applications in the modification of biomaterials.

It is noted that the term “about” may be utilized herein to represent the inherent degree of uncertainty that may be attributed to any quantitative comparison, value, measurement, or other representation. This term is also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A substrate having attached thereto, or associated therewith, one or more poly(2-alkyl-2-oxazine) or copolymers containing at least one poly(2-alkyl-2-oxazine), wherein alkyl is a hydrocarbon of formula CₓH₂ₓ₊₁ where x is greater than or equal to 1.

2. The substrate of claim 1, wherein the one or more poly(2-alkyl-2-oxazine) is selected from the group consisting of poly(2-methyl-2-oxazine), poly(2-ethyl-2-oxazine) and copolymers containing poly(2-methyl-2-oxazine), poly(2-ethyl-2-oxazine), or both.

3. The substrate of claim 1, wherein the substrate has attached thereto or associated therewith a copolymer of at least one poly(2-alkyl-2-oxazine) and a poly(2-alkyl-2-oxazoline).

4. The substrate of claim 3, wherein the copolymer comprises at least 50 mol.% poly(2-alkyl-2-oxazine).

5. The substrate of claim 3, wherein the poly(2-alkyl-2-oxazine) is selected from the group consisting of poly(2-methyl-2-oxazine) and poly(2-ethyl-2-oxazine).

6. The substrate of claim 3, wherein alkyl in the poly(2-alkyl-2-oxazine) and the poly(2-alkyl-2-oxazoline) is selected from the group consisting of methyl and ethyl.

5. The substrate of claim 1, wherein the substrate is selected from the group consisting of polymeric supports, metal supports, metal oxide supports, glass supports, quartz supports, and silicon supports.

6. The substrate of claim 1, wherein said substrate is a medical implant, a biomedical implant, a drug delivery carrier, a biosensor, or a marine coating.
